(19) United States
(12) Patent Application Publication
(10) Pub. No.: US 2016/0296736 A1
(43) Pub. Date: Oct. 13, 2016

(54) VENTRICULOPERITONEAL SHUNT WITH DISTAL BALLOON

(71) Applicant: University of Iowa Research Foundation, Iowa City, IA (US)

(72) Inventors: Oliver Elias Flouty, Iowa City, IA (US); Kingsley Otsioren Abode-Iyamah, Iowa City, IA (US); Matthew A. Howard, Iowa City, IA (US); Jeremy D. Greenlee, Iowa CIty, IA (US)

(21) Appl. No.: 15/093,864

(22) Filed: Apr. 8, 2016

Related U.S. Application Data
(60) Provisional application No. 62/144,496, filed on Apr. 8, 2015.

(51) Int. Cl. A61M 27/00

(52) U.S. Cl. CPC A61M 27/006; A61M 2025/1079

(57) ABSTRACT

A ventriculoperitoneal shunt has a ventricular catheter and a peritoneal catheter, with a shunt valve connected in line between the catheters to define a drainage patch for cerebrospinal fluid from the brain ventricle to the peritoneal cavity. An inflatable balloon on the peritoneal catheter precludes migration or displacement of the catheter in the peritoneal cavity, thereby eliminating or minimizing corrective revision surgery.

### CROSS REFERENCE TO RELATED APPLICATIONS

This application claims priority under 35 U.S.C. §119 to provisional application U.S. Ser. No. 62/144,496 filed Apr. 8, 2015, all of which is herein incorporated by reference in its entirety.

### BACKGROUND OF THE INVENTIONS

Certain medical conditions, such as head injuries, congenital malformation, bleeding from a blood vessel in the brain, idiopathic intracranial hypertension and meningitis, can produce increase in intracranial pressure (ICP) by preventing normal drainage of cerebrospinal fluid (CSF). Such CSF buildup typically occurs in the ventricles inside the brain, which cause the ventricles to enlarge and compress the brain against the skull, leading to increased ICP. This increase in ICP can cause serious neurological problems, including death. This condition is known medically as hydrocephalus.

In conditions where the cerebrospinal fluid must be drained for significant periods of time, a ventriculoperitoneal (VP) shunt is typically used. The VP shunt consists of a proximal catheter extending into the brain ventricle, with a valve attached to the catheter to control the amount of CSF being drained from the ventricle. A second catheter is attached to the distal end of the valve and tunneled beneath the skin into the peritoneal cavity wherein the CSF is reabsorbed.

The VP shunt procedure is done as follows:

An area of hair on the head is shaved. This may be behind the ear or on the top or back of the head. The surgeon makes a U-shape cut behind the ear. Another small surgical cut is made in the belly. A small hole is drilled in the skull. A thin tube or catheter is passed into a ventricle of the brain. This can be done with or without a computer as a guide. It can also be done with an endoscope that allows the surgeon to see inside the ventricle. Another catheter is placed under the skin behind the ear. It is sent down the neck and chest, and usually into the belly area or alternative 14, into the chest area. The doctor may make a small cut in the neck to help position it. A valve (fluid pump) is placed underneath the skin behind the ear. The valve is connected to both catheters. When extra pressure builds up around the brain, the valve opens, and excess fluid drains through the catheter into the belly or chest area. This helps lower intracranial pressure.

According to medical statistics, VP shunts are the most common neurological procedure performed in the United States, yet these VP shunts have a 40% failure rate within two years, primarily due to catheter migration and/or infection. For example, the distal catheter extending into the abdominal cavity can accidentally back out from the peritoneum, which causes the CSF to collect under the skin without reabsorption. Catheter migration or pull out is particularly a problem in children due to their growth, and in obese patients. Such pull-out of the catheter requires another surgery to re-secure the distal catheter into the peritoneal cavity. The average cost for such corrective surgery is $36,000. The corrective surgery also exposes the patient to the other risks associated with any surgery.

Accordingly, a primary objective of the present invention is the provision of a ventriculoperitoneal shunt having a distal catheter which will not accidentally pull from the peritoneal cavity.

A further objective of the present invention is the provision of a VP shunt having an inflatable balloon to retain the catheter in position in the peritoneal cavity.

Another objective of the present invention is the provision of a VP shunt having a dual lumen distal catheter to permit inflation and deflation of a balloon and draining of CSF fluid from the brain.

Another objective of the present invention is a provision of a VP shunt having a distal catheter wherein the valve also functions to allow inflation and deflation of a balloon.

Another objective of the present invention is the provision of an improved VP shunt which can be quickly and easily secured in the peritoneal cavity.

A further objective of the present invention is the provision of a method for draining cerebrospinal fluid from a brain ventricle without risk of malfunction due to catheter migration.

Still another objective of the present invention is the provision of a method for retaining a VP shunt in the peritoneal cavity by inflating a balloon on the distal catheter.

Another objective of the present invention is a provision of an improved VP shunt which is economical to manufacture, and safe to use.

These and other objectives will become apparent from the following description of the invention.

### BRIEF DESCRIPTION OF THE DRAWINGS

FIG. 1 is a schematic view of a patient having the ventriculoperitoneal shunt of the present invention.

FIG. 2 is an enlarged perspective view of a dual lumen distal catheter for inflation of the balloon, according to the present invention.

FIG. 3 is another perspective schematic showing a dual lumen distal catheter and balloon inflation syringe.

FIG. 4 is a perspective schematic showing a dual lumen distal catheter with the shunt valve.

FIG. 5 is a sectional view of the dual lumen distal catheter with the shunt valve inserted therein.

FIG. 6 is a perspective schematic view showing the shunt valve installed in the distal catheter.

FIG. 7 is a sketch showing the deflated balloon with radiopaque material.

FIG. 8 is a sketch showing the inflated balloon with radiopaque material.

FIG. 9 is a perspective view of an alternative embodiment of the distal catheter with an external balloon inflation tube.

FIG. 10 is a perspective view of an alternative embodiment of the distal catheter with the valve acting as the site for inflation of the balloon and connection from the valve to the inflation lumen.

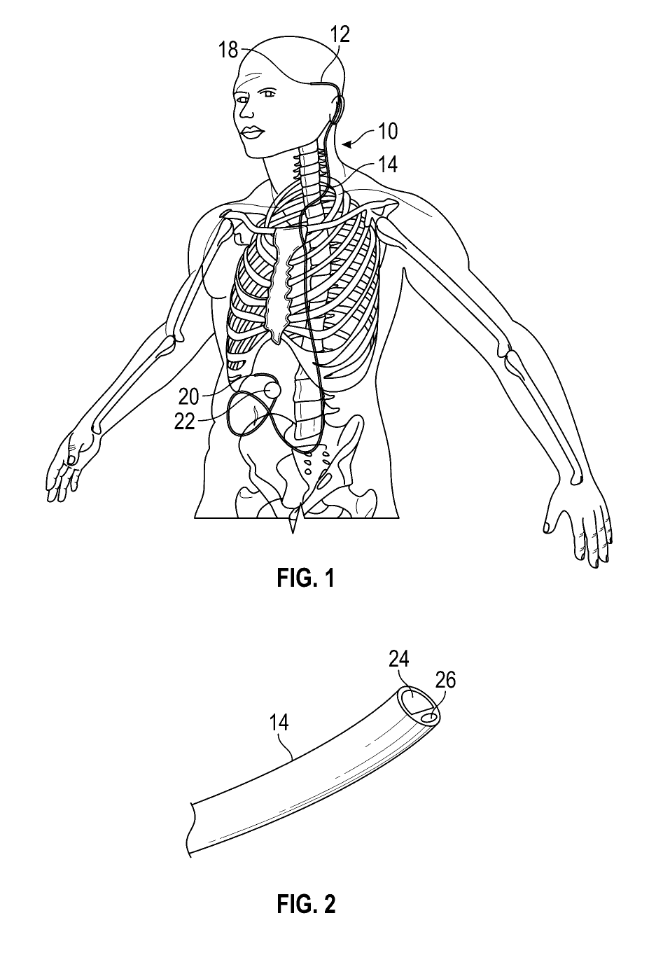

### DETAILED DESCRIPTION OF THE INVENTION

The ventriculoperitoneal shunt 10 of the invention includes a proximal or ventricular catheter 12 and a distal or peritoneal catheter 14. A shunt valve 16 connects the adjacent ends of the catheters 12, 14. The proximal or free end 18 of the ventricular catheter 12 is adapted to be inserted into the brain ventricle, while the distal or free end 20 of the peritoneal catheter 14 is adapted to be inserted into the peritoneal cavity, with the catheter 12 tunneling beneath the skin between the opposite ends 18, 20. The catheters, when connected to the valve 16, form a continuous catheter drain path from the brain ventricle to the peritoneal cavity.

The above described structure for the VP shunt 10 is conventional.

The present invention improves the conventional VP shunt 10 by incorporating an inflatable balloon 22 on the distal end of the catheter 14. The balloon resides within the peritoneal cavity, and is surgically implanted in a deflated condition. During surgery, the balloon 22 is inflated to maintain the end 20 of the catheter 14 within the peritoneal cavity and preclude accidental pullout of the catheter 14 from the cavity.

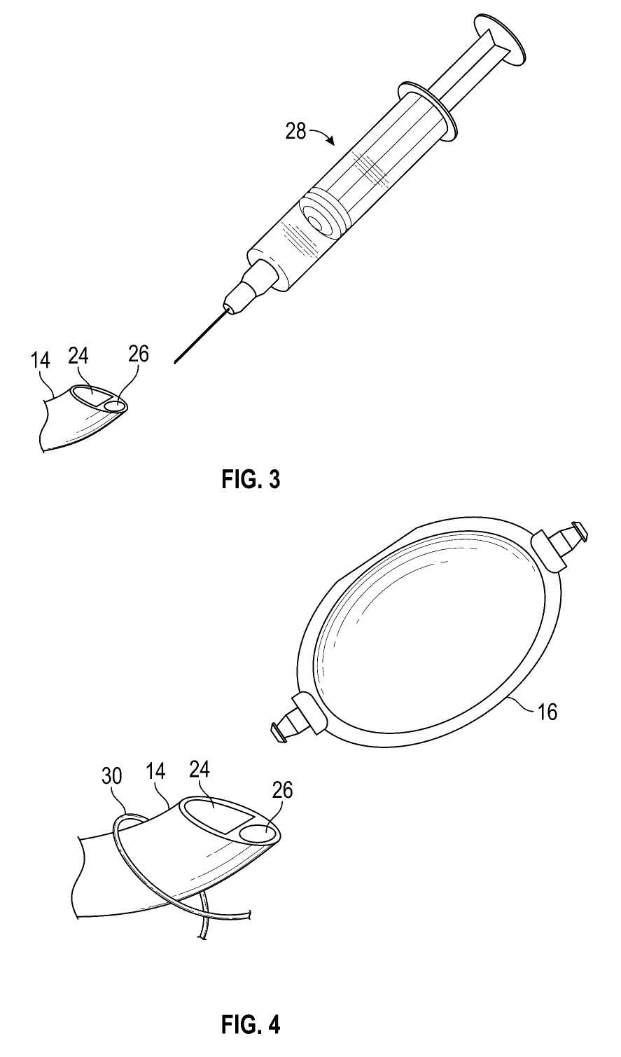

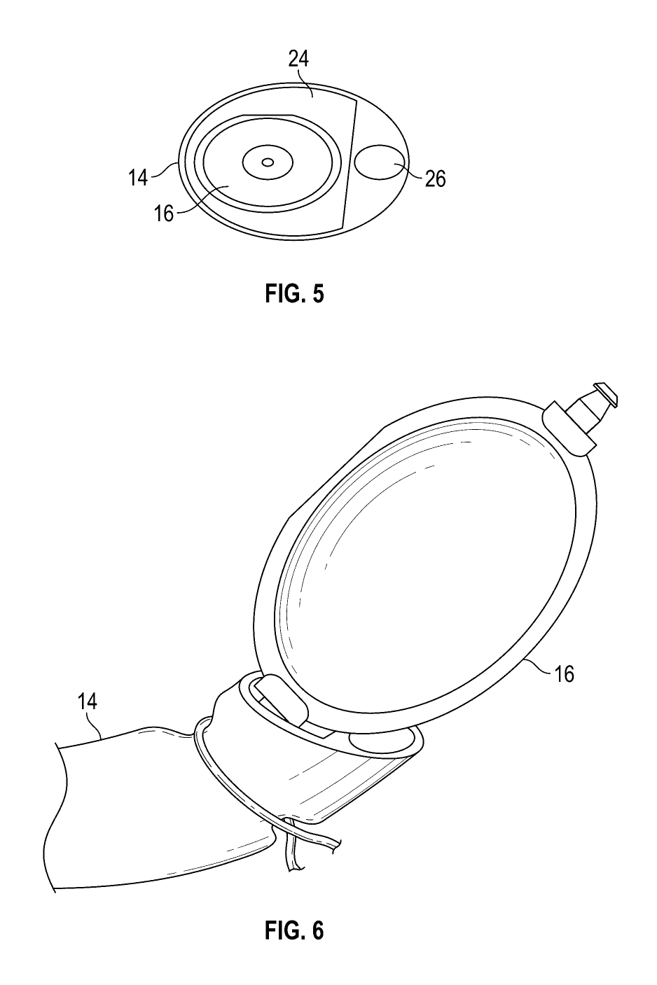

In a preferred embodiment, the distal catheter 14 has a double lumen construction, as shown in FIGS. 2-4, with a CSF tube 24 and a balloon inflation tube 26. The proximal or upper end of the catheter 14 is located in a convenient position on the patient's body, such as in the neck area, for easy access to the inflation tube 26. A syringe 28 can be used to inflate the balloon 22 with saline solution. In an alternative embodiment, shown in FIG. 9, the inflation tube 26 is external to the CSF tube 24.

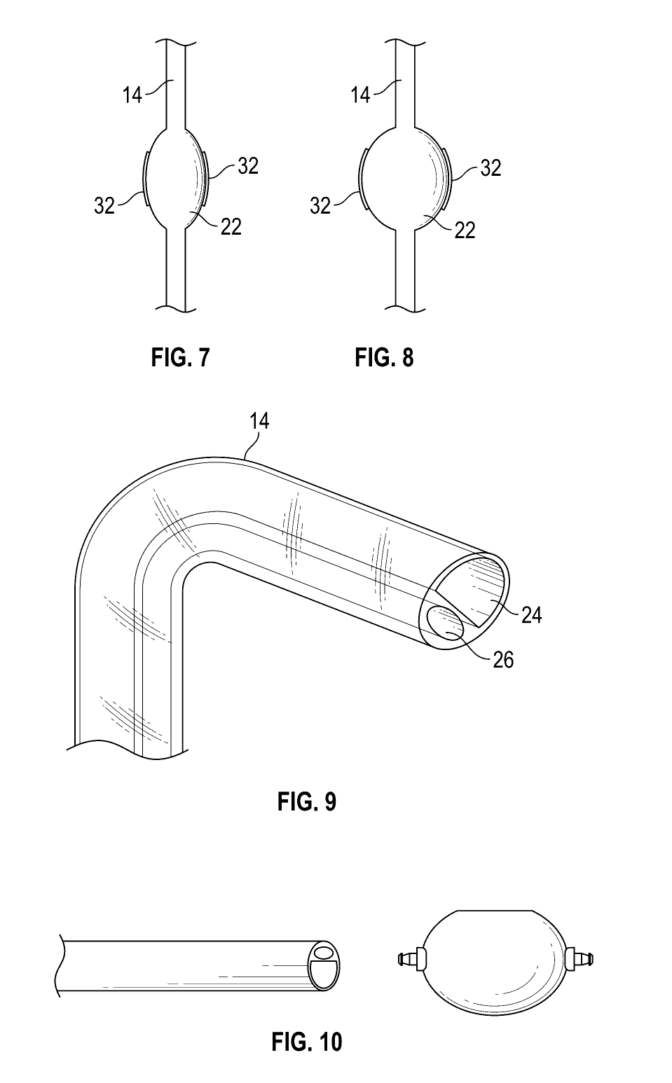

In a preferred embodiment, the balloon 22 is coated on opposite external sides with a radiopaque material 30, as shown in FIGS. 7 and 8. The radiopaque markers 30 can be imaged, using x-ray or other known equipment, so as to allow a medical professional to confirm the inflated and deflated condition of the balloon, based upon the distance between the markers 30.

For implementation of the VP shunt 10, the distal end 20 of the catheter 14 is surgically implanted into the peritoneal cavity and then the catheter is tunneled beneath the skin to a proximal position. The balloon 22 is then inflated with sterile saline solution from the syringe 28. The catheter 14 is then clamped to prevent loss of the saline solution. The valve 16 is then installed in the catheter 14 and secured with a clamp 30, such as a tevdek tie, which also helps prevent saline loss. After the valve 16 is connected to the catheter 14, the first clamp is removed so that the CSF tube is open. The proximal catheter 12 is then surgically implanted into the brain ventricle and connected to the valve 16 so that CSF can drain from the ventricle to the peritoneal cavity.

Thus, the inflatable balloon 22 on the distal catheter 14 will decrease catheter migration, without significantly changing the surgical implantation time or process and will eliminate or minimize revision surgery to reattach or reposition a displaced distal catheter.

More particularly, the improved ventriculperitoneal shunt of the present invention minimizes or eliminates catheter migration when the balloon is inflated. This shunt does not significantly change the surgery routine or time. The balloon VP shunt also saves significant costs by reducing revision surgery, which benefits the patient by saving time and money, while decreasing the likelihood of re-hospitalization; saves the hospital money by avoiding post procedure complications and re-hospitalization, and thereby freeing valuable resources for other uses; saves the physician time; and provides financial savings for insurance companies. The VP shunt can be used on pediatric patients and adult patients.

The invention has been shown and described above with the preferred embodiments, and it is understood that many modifications, substitutions, and additions may be made which are within the intended spirit and scope of the invention. From the foregoing, it can be seen that the present invention accomplishes at least all of its stated objectives.

What is claimed is:

1. A ventriculoperitoneal shunt, comprising:
a catheter with a proximal end adapted to be inserted into a ventricle of the brain and a distal end adapted to be tunneled under the skin into the peritoneal cavity;
a shunt valve between the catheter ends;
an inflatable balloon adjacent the distal end; and
the catheter including an inflation tube having a first end terminating in the balloon and a second end at a proximal location upstream from the balloon.

2. The ventriculoperitoneal shunt of claim 1 wherein the catheter is a double lumen with one of the lumens permitting fluid flow from the brain to the peritoneal cavity and the other lumen constituting the inflation tube.

3. The ventriculoperitoneal shunt of claim 1 wherein the balloon includes radiopaque material to indicate an inflated status and a deflated status of the balloon.

4. The ventriculoperitoneal shunt of claim 1 wherein the shunt valve is removably connected to the catheter.

5. A ventriculoperitoneal shunt, comprising:
a ventricular catheter;
a peritoneal catherer;
a shunt valve connected to the ventricular and peritoneal catheters to form a continuous catheter drain path; and
an inflatable balloon on the peritoneal catheter.

6. The ventriculoperitoneal shunt of claim 5 wherein the peritoneal catheter includes an inflation tube to supply a fluid to the balloon for inflation of the balloon.

7. The ventriculoperitoneal shunt of claim 6 wherein the inflation tube is internally formed in the peritoneal catheter.

8. The ventriculoperitoneal shunt of claim 6 wherein the inflation tube is externally formed on the peritoneal catheter.

9. The ventriculoperitoneal shunt of claim 5 wherein the peritoneal catheter has double lumens.

10. The ventriculoperitoneal shunt of claim 5 wherein the balloon has a radiopaque coating.

11. The ventriculoperitoneal shunt of claim 5 wherein the catheters are detachably connected to the shunt valve.

12. The ventriculoperitoneal shunt of claim 5 wherein the balloon is downstream from the valve.

13. A method for draining cerebrospinal fluid from a brain ventricle, comprising:
inserting a distal end of the catheter into the peritoneal cavity;
inflating a balloon on the catheter inside the peritoneal cavity to retain the catheter in the peritoneal cavity;
inserting a proximal end of a catheter into the brain ventricle; and
opening a valve on the catheter to permit fluid flow from the brain ventricle to the peritoneal cavity.

14. The method of claim 15 wherein the balloon is inflated from a proximal location.

15. The method of claim 13 wherein the balloon is inflated through a lumen on the catheter.

16. The method of claim 13 wherein the balloon is inflated with a saline solution.

17. The method of claim 13 further comprising imaging a radiopaque layer on the balloon to determine the balloon inflation status.

18. The method of claim 13 further comprising monitoring the balloon via imaging.

19. The method of claim 13 further comprising deflating the balloon before removing the catheter from the peritoneal cavity.
